(19) United States
(12) Patent Application Publication
(10) Pub. No.: US 2021/0187206 A1
(43) Pub. Date: Jun. 24, 2021

(54) SYRINGE DESTRUCTION

(71) Applicant: Needlesmart Limited, Liverpool, Merseyside (GB)

(72) Inventor: Clifford Ian Kirby, Sefton Village, Merseyside (GB)

(21) Appl. No.: 16/973,973

(22) PCT Filed: Jul. 8, 2019
(86) PCT No.: PCT/EP2019/068278

§ 371 Date: Dec. 10, 2020

(30) Foreign Application Priority Data
Jun. 12, 2018 (GB) 1809626.3

(51) Int. Cl. A61M 5/32

(52) U.S. Cl. CPC A61M 5/3278; A61M 2005/3283; A61M 2005/3209; A61M 2005/3206; A61M 2205/332

(57) ABSTRACT

An apparatus for destroying syringes-needle assemblies is disclosed, the invention comprising a cradle for holding the syringe and a hub grip for gripping the hub of a needle affixed to the syringe. The apparatus comprises means for destroying the needle for example, by passing a current through it to soften/melt it, whilst at the same time, applying an axial compressive force to blunt and compress the needle. The hub grip is moveable relative to the cradle so that the needle can be pulled, unscrewed or otherwise detached from the syringe. The apparatus is preferably automated, and thus enables the destroyed needle and syringe to be separated, and therefore disposed of in separate waste streams without manual intervention.

This invention relates to an apparatus and method for the safe destruction of used syringes.

Syringes are used ubiquitously in medicine and other applications, and generally comprise a syringe main body and a hypodermic needle affixed to the syringe main body. The syringe main body typically comprises a cylinder and a plunger, whicn acts as a piston to force and/or draw fluids through an opening at the end of the cylinder. The opening is typically formed as a spigot, to which the hypodermic needle is sealingly affixed. Meanwhile, the hypodermic needle comprises a hub, which connects to the syringe's spigot, and a tubular needle with a sharp tip, which protrudes therefrom. The hub can be connected to the spigot by frictional engagement, via a screw-threaded connection, a bayonet-type connection, etc.

The syringe can be used for injecting or withdrawing liquid substances into, or from, a patient's circulatory system in a manner that is well understood. The needle for a hypodermic syringe typically comprises a fine metal tube with a sharp end, which is adapted, in use, to pierce the patient's skin and, in many cases, enter a blood vessel for the delivery of a liquid medicament, or the extraction of a fluid, as the case may be. Once used, a hypodermic needle needs to be disposed of safely because the needle becomes contaminated during use. If a medical practitioner inadvertently suffers a “needle stick injury”, i.e. if he/she pierces their own skin with a contaminated needle, then there is a risk of disease/infection being passed from the patient to the medical practitioner.

Various methods of disposing of hypodermic needles are known, for example, the use of “sharps bins” into which used/contaminated needles are placed. A sharps bin generally comprises a pierce-resistant main body, which prevents, or inhibits, the contaminated needle points from coming into contact with people. The sharps bin usually has an obstructed opening that permits needles to be placed into the main body, but which prevents or restricts a user from placing their hand or other body parts into the main body.

Other methods of hypodermic needle destruction are disclosed in, for example, published PCT application No. WO2015/011443. In this disclosure, the sharp end of the needle is inserted into a device, whereupon a current is passed through the needle to soften/melt its tip. Axial compression of the needle, whilst in the softened/melted state, causes the needle tip to be deformed into a blunt ball, which prevents the formerly sharp tip from posing a needlestick injury. Moreover, hypodermic needle destruction devices of this general type also have the advantage of heating the needle to or near its melting point, the heat of which destroys any viruses, bacteria and contaminants. Devices of this general type therefore offer two-fold protection, that is: blunting and sterilising the needle.

It will be appreciated from the foregong, that devices and methods for the disposal of hypodermic needles are well-known. However, once “destroyed” there still exists a potential problem with waste management because even though the needle has been blunted/sterilised, it is still attached to the syringe.

As such, a further problem exists in relation to the waste stream for used syringes because of the materials of manufacture, namely the metal of the needle, the epoxy resin/piastic of the hub, as well as the plastic of the main body of the syringe. When these materials are “co-mingled”, recycling of these various components is extremely difficult—especially where a biohazard is involved as well.

The needle and syringe can therefore benefit from being treated/processed via different waste streams (e.g. plastics vs metals vs non-recyclables), and so it is often desirable to disassemble the syringe, that is to say, to separate the blunted/sterilised needle from the syringe so that the two can be disposed of separately.

Whilst needle destruction devices, such as that described in published PCT application No. WO2015/011443 are known, there still exists a need to further “process” the syringe by disassembling it, which according to the state of the art, requires a user to manually separate the needle from the syringe. Further, even though devices such as that described in published PCT application No. WO2015/011443 can destroy the needle, there is nevertheless a finite risk of needlestick injury whilst handling the un-treated syringe prior to destruction, and whilst manipulating it into the needle destruction device.

A need therefore exists for a solution to one or more of the above problems, which this invention aims to provide.

Various aspects of the invention are set forth in the appended independent claim or claims. Optional or preferred features of the invention are set forth in the appended dependent claims.

According to an aspect of the invention, there is provided: a syringe processing apparatus for syringe disposal, the syringe comprising a main body and a hypodermic needle affixed thereto, the main body comprising a spigot to which a hub that supports a needle part of the hypodermic needle is affixed, the syringe processing apparatus comprising: a needle destruction assembly for destroying the needle part of the hypodermic needle, the needle destruction assembly comprising a conductive clamping electrode adapted to clamp the needle part of the hypodermic needle at a point between the hub and its tip, a conductive tip electrode for contacting the tip of the needle part of the hypodermic needle, the tip electrode being driven to move coaxially with an axis of the needle part of the hypodermic needle, means for applying a voltage between the clamplng electrode and the tip electrode as the tip electrode is advanced towards the hub, such that resistive heating of the needle occurs in conjunction with axial compression exerted by the tip electrode on the needle, which softens/melts the tip of the needle, thereby blunting and sterilising the needle tip, the apparatus further comprising: a cradle for supporting and holding the main body and being driven to move the main body along a locus that is parallel with the said axis; and a hub grip assembly for gripping the hub of the hypodermic needle, which comprises an annular grip part adapted to engage the hub of the hypodermic needle when the cradle is driven along the said axis, wherein the cradle and hub grip assembly are relatively moveable so as to separate the hub of the hypodermic needle from the spigot of the mam body.

By making the cradle and hub grip assembly relatively moveable, it is possible to automate the disassembly of the hypodermic needle from the syringe. In certain embodiments, a controller is provided that coordinates the relative movement so as to overcome frictional engagement between the hypodermic needle and the spigot of the syringe and/or to unscrew the hypodermic needle from the spigot of the syringe; and/or disconnect a bayonet-type connection between the hypodermic needle and the spigot of the syringe.

Suitably, the needle destruction assembly further comprises a containment cylinder, which surrounds the needle part of the hypodermic needle, and within which the tip electrode is arranged to move. The containment cylinder suitably prevents or inhibits the needle part of the hypodermic needle from bowing or buckling as the axial compressive force is applied thereto.

The containment cylinder may additionally comprise a heater coil, for pre-heating the containment cylinder or a needle located therein. This can be useful for driving-off moisture or liquids within the containment cylinder and/or within the bore of the needle part of the hypodermic needle prior to the application of the voltage and axial compression. If moisture is present during the heating compression process, vapour droplets can form, which can adversely affect the formation of a blunt ball of metal at the needle tip.

In certain embodiments of the invention, there is provided an induction coil surrounding the needle, which carries an electric current that incuces, in use, a current in the needle. Resultant eddy current heating of the needle can be used to assist the melting process via resistive/Ohmic heating.

The voltage applied between the tip electrode and the clamping electrode can be of any suitable type, such as a DC voltage, an AC voltage, and RF voltage or any other suitable voltage. A control/feedback system is suitably provided for coordinating the application of the voltage and the axial compression force. As the needle tip melts, it will tend to recede from the tip electrode, thereby either breaking the circuit and/or by increasing the resistance in the circuit. As such, a high speed feedback circuit ts suitably provided, which controls the applied voltage end displacement of the tip electrode so as to maintain the tip electrode in contact with the tip, whilst maintaining the current within the needle (between the clamping and tip electrodes) within specified parameters.

The tip electrode suitably comprises a formation, such as a recess, for centralising the tip of the needle part of the hypodermic needle with the centre of the tip electrode.

The tip electrode is driven to move coaxially with an axis of the needle part of the hypodermic needle and this can be by way of a pulley belt arrangement, a rack and pinion, a worm screw, a linear actuator, or by any suitable means.

The cradle supports and holds the main body of the syringe and is driven to move the main body along a locus that is parallel with the said axis. Movement of the cradle can be by way of any suitabie means, such as a pulley belt arrangement, a rack and pinion, a worm screw, and/or a linear actuator.

In one embodiment, the cradle comprises a main body, which is shaped to receive the syringe main body. The shape suitably comprises end abutments, which engage the distal and proximal ends of the syringe main body, thereby preventing or inhibiting axial displacement of the syringe relative to the cradle.

In certain applications, the hypodermic needle is not coaxial with the centreline of the syringe main body, and this can make it difficult to maintain the required axial alignment of the various parts of the syringe processing apparatus. In certain embodiments, therefore, means is provided for adjusting the cradle configuration such that the hypodermic needle automatically aligns with a datum line, which datum line can be brought into alignment with the axis of the syringe processing apparatus.

In one embodiment, a centraliser is provided, which comprises a tapered aperture that “catches” the hypodermic needle as a syringe is placed into the syringe processing apparatus. The taper is suitably configured to cause the needle part of the hypodermic needle to align with an axis of the centraliser, and be retained thereby, for example, by gravity. With the needle part of the hypodermic needle (with the syringe attached thereto) temporarily retained by the centraliser, adjustable grip means of the cradle can be used to grip the syringe main body before the configuration is effectively locked-in. Now, the centraliser can be removed, and the alignment of the needle part of the hypodermic needle is fixed relative to a datum of the cradle, which conveniently aligns with the axis of the syringe processing apparatus, and so now the apparatus is correctly set-up and aligned.

The hub grip assembly grips the hub of the hypodermic needle by way of an annular grip part adapted to engage the hub of the hypodermic needle when the cradle is driven along the said axis. The annular grip part suitably comprises a resiliently deformable annulus, for example, manufactured from an elastomeric material, which frictionaily engages the hub as it is axially inserted into it.

The cradle and hub grip assembly are relatively moveable so as to separate the hub of the hypodermic needle from the spigot of the mam body. This can be by way of axial displacement of the cradle relative to the hub grip. This configuration may be used to disconnect a frictionally-engaged hub from the syringe's spigot. In this case, the frictional engagement of the hub with the hub grip should be greater than the frictional engagement of the hub with the syringe's spigot.

However, in certain embodiments, the hub grip can be configured to rotate about the said axis, for example, by driving the annual hub grip using a ring gear driven by an electric motor. By such means, the hub can be rotated (about the said axis) relative to the spigot of the syringe. Rotating/oscillating the hub relative to the spigot can often be sufficient to separate it from the spigot.

In certain embodiments, the rotation/oscillation of the hub relative to the spigot is coordinated with axial displacement of the hub relative to the spigot. A controller may be used for this purpose, which measures and/or controls the forces and/or displacements that occur. It will be appreciated that the coordinated use of relative rotational and relative axial displacement can be used to unscrew a screw-threaded hub from the spigot and/or to disconnect a bayonet connected hub from the spigot.

The control system could comprise a logic circuit, which attempts to disconnect the hub from the spigot via various methods, such as: axial displacement (friction-fitted hub); axial displacement+rotational oscillation (tightly friction-fitted hub); axial displacement+rotation (screw-fitted hub); axial rotation+linear displacement (bayonet-connected hub). By measuring the forces and displacements, the control system could be configured to assess whether each method has worked, before proceeding to another method if not.

Syringe destruction devices in accordance with the invention are shown in the accompanying drawings in which:

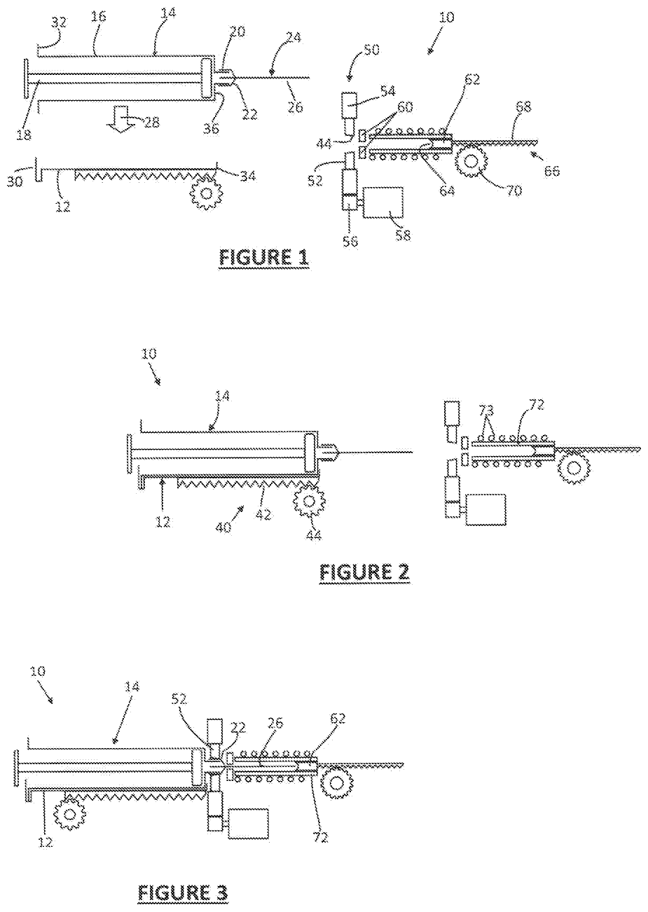

FIG. 1 is a schematic side view of a first embodiment of an apparatus in accordance with the invention;

FIGS. 2, 3, 4, 5, 6, 7, 9 and 10 are a sequence showing the operation of the apparatus shown in FIG. 1;

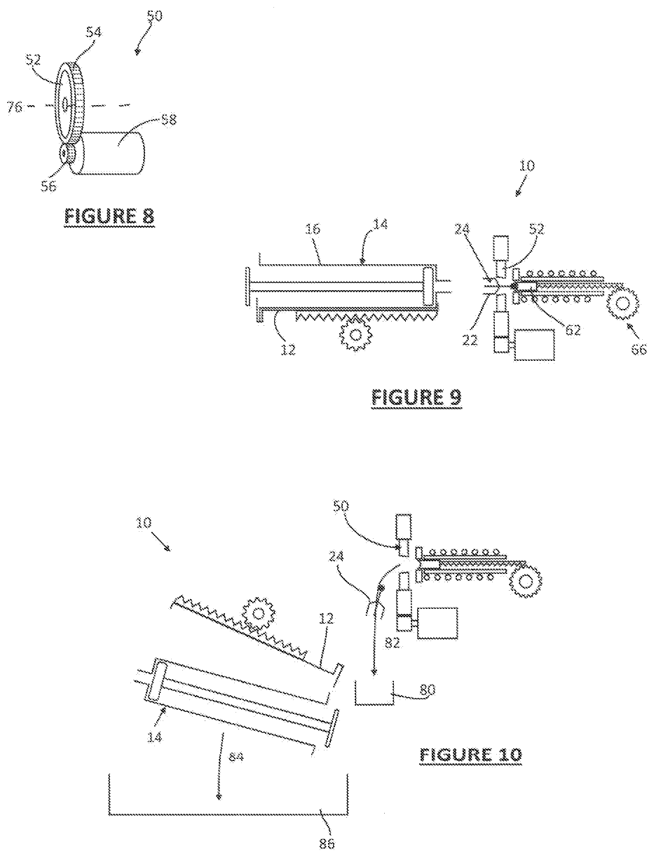

FIG. 8 is a partial perspective view of a feature shown in FIG. 7;

FIG. 11 shows an alternative embodiment of the invention comprising a centraliser; and

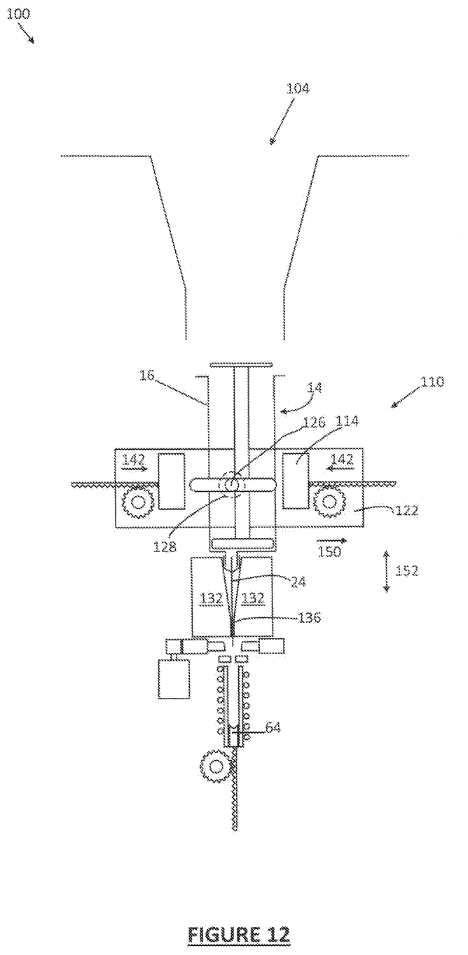

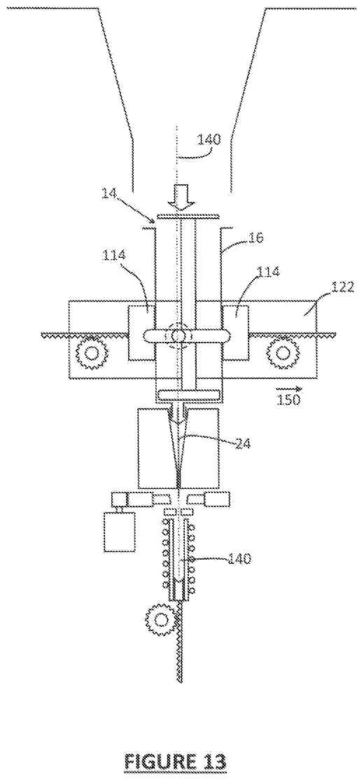

FIGS. 12-14 are a sequence showing the operation of the apparatus shown in FIG. 11.

A syringe disposal device 10 in accordance with the invention comprises a cradle 12 adapted to receive, and retain, a syringe 14. The syringe 14 has a main body 16 and an internal plunger 18, and the main body 16 has a spigot 20, to which the hub 22 of a hypodermic needle 24 is fixed. The hypodermic needle 24 also comprises a needle part 26, which is attached to the hub 22 using a grout or an adhesive.

The apparatus 10 has an opening (not shown) through which the syringe 14 can be placed, where it drops 28 down into the cradle 12. The cradle 12 has a rear abutment 30, which engages a flange 32 of the main body 16 of the syringe 14; a front abutment 34, which engages the front face 36 of the syringe main body 16.

Once inserted, as shown in FIG. 2, the syringe 34 sits in, and is retained by, the cradle 12. The cradle 12 is mounted on a linear actuator 40, which in the illustrated embodiment, comprises a rack 42 and pinion 44 arrangement, although other types of linear actuator 44 are within scope of the invention, for example, a pulley system and/or a worm screw. Regardless, the linear actuator 40 allows the cradle 12, and hence the syringe 14 to be advanced, to the position as shown in FIG. 3 of the drawings.

Referring back to FIG. 1 of the drawings, the apparatus 10 comprises a hub engagement part 50, which has an annulus 52, with a tapered through-hole 54, frictionally-engages the hub 22 when inserted therein, as shown in FIG. 3. The rubber anulus 52 is contained within an annular ring gear 54, which meshes with a pinion gear 56, driven by a motor 58. The operation of the hub-gripping part 50 is described in greater detail below.

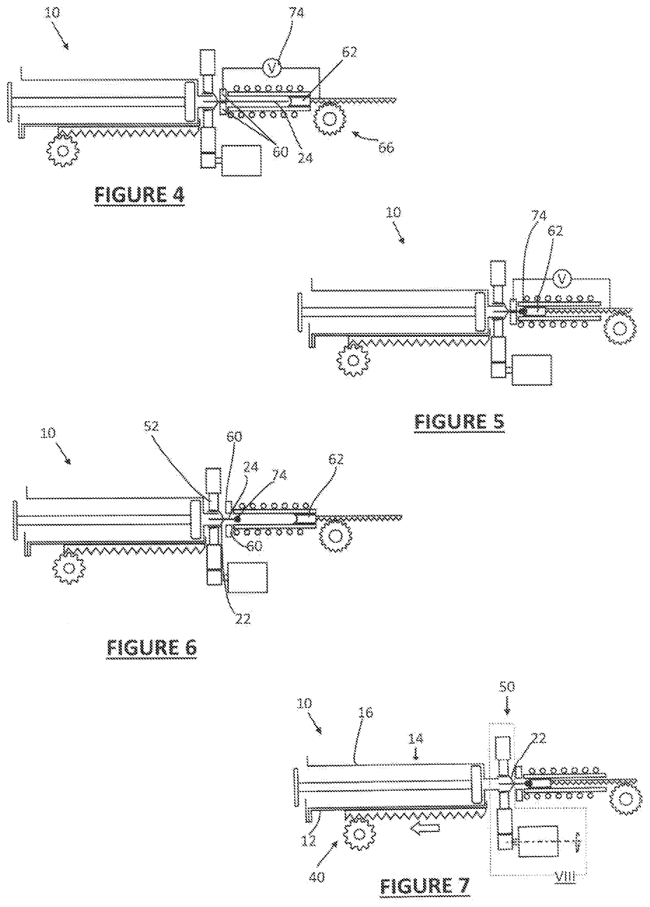

The apparatus 10 also comprises a set of clamping electrodes 60, which are made of metal, and which can be separated, as shown in FIG. 1 to enable the needle 26 to pass therebetween; or moved towards one another, as shown in FIGS. 4 and 5 of the drawings, so as to clamp the needle 26 therebetween and form an electrical contact therewith.

The apparatus 10 also comprises a tip electrode 62, which is also made of metal, and which has a dish-shaped end 64, which serves to centralise the tip electrode 62 on the needle 26 tip, in use.

The tip electrode 62 is also mounted on a linear actuator 26 which in the illustrated embodiment, comprises a rack 68 and a pinion 70 arrangement, although other linear actuators may equally be used, such as pulley belt system and/or a worm screw.

Referring back to FIG. 3 of the drawings now, the cradle 12 has been advanced such that the hub 22 is fictionally engaged in the rubber anulus 52 and the needle 26 protrudes to a containment cylinder 72 with the tip electrode 62 in a retracted position. An induction and/or heater coil 73 surrounds the containment cylinder 72.

Referring now to FIG. 4 of the drawings, the clamping electrodes 60 have been moved together so as to clamp the needle 24 therebetween. A voltage 74 is then applied between the clamping electrodes 60 and the tip electrode 62 such that an electric current passes through the needle 24. Due to resistive/ohmic heating, the needle 24 softens as it heats, and at the same time, the tip electrode's linear actuator 66 is driven so as to push the tip electrode, along the axis of the needle 24, towards the hub 22.

The end result is shown in FIG. 5 of the drawings where the tip electrode 62 has been fully advanced and the needle 24 tip has been blunted and melted into a round ball 74. The process of heating/melting the needle tip 74 serves to sterilise it as the temperature involved is above that at which bacteria, viruses and other pathogens can survive; whilst at the same time blunting the tip, and thereby removing any risk of a “needle stick” injury.

Once the needle 24 has been blunted and sterilised, as shown in FIG. 6, the voltage 74 can be removed, the clamping electrodes 60 moved apart and the tip electrode 62 retracted, if necessary. At this point, the hub 22 is still gripped by the anulus 52.

Referring to FIGS. 7 and 8 of the drawings, it can be seen that the hub grip assembly 50 is now manipulated, by driving the motor 58, so as to turn the pinion gear 56 and thus the ring gear 54, which thereby rotates the hub about an axis 76, which is co-axial with the longitudinal axis of the needle 24. At the same time, the cradle's linear actuator 40 is driven so as to slowly retract the cradle 12, and hence the main body 16 of the syringe 14 such that the rotation of the hub 22 relative to the fixed orientation of the syringe 24 unscrews or releases the hub 22, and hence the needle 24 from the main body 16.

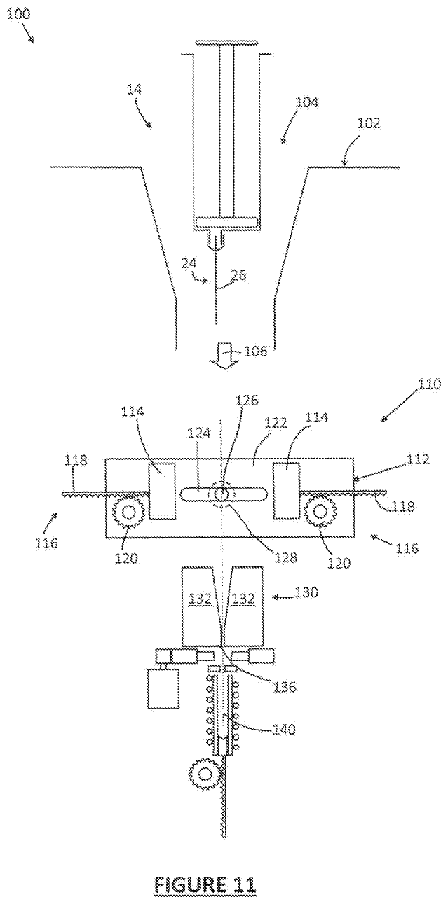

The result is shown in FIG. 9 of the drawings, where it can be seen that the syringe body 16 has been fully-separated from the hub 22 of the hypodermic needle 24. In order to disengage the hub 22 from the anulus 52, the tip electrode 62 can be advanced again, using its drive mechanism 66, so as to axially displace the hub 22 from the rubber anulus 52, thereby releasing it. Referring to FIG. 10, a needle waste receptacle 80 is provided to catch the hypodermic needle 24, after processing, as it falls away from the hub grip assembly 52. Also shown in FIG. 10, is the cradle 12 being inverted so that the remainder of the syringe 14 can fall away 84 into a further waste receptacle 86.

It will be appreciated from the foregoing description that the syringe/needle 14/24 can be safely destroyed and disassembled and its parts separated into separate waste streams, 80, 86. Due to the provision or a cradle, it is not necessary for an operator to hold the syringe 24/needle 24 once it has been used: It can simply be dropped into the apparatus 10, where the destruction process proceeds automatically, without further user intervention.

A further embodiment 100 of the syringe processing apparatus in accordance with the invention comprises an outer housing 102 with an aperture 104 through which a syringe can be dropped 6, in use. Identical reference signs have been used in FIGS. 11 to 14 to those used in FIGS. 1 to 8, to identify identical features and thus avoid unnecessary repetition herein.

The main difference between the embodiment 100 shown in FIGS. 11-14 of the drawings, and that shown in FIGS. 1-10 of the drawings, is the addition of a “self centralising” cradle 110. The self-centralising cradle 110 comprises a main cradle part 112, which carries a pair of adjustable grips 114, which are driven to move by actuators 116, in this case, rack 118 and pinion 120 devices. The adjustable grips 114 are mounted on a plate 122 which has a slotted aperture 124, which permits the plate 122 to move left/right (as shown in the drawing). A mount 126 locates within the slotted aperture 124 and allows the plate 122 to move left/right (as shown in the drawing) as previously described, and a locking ring 128 enables the position of the plate 122 relative to its mount 126 to be locked.

As shown in FIG. 12 of the drawings, when a syringe 14 is dropped into the device 100 via the aperture 104, the needle 24 is guided by the tapered centraliser parts 132 such that the needle 24 falls into the space 136 between the centraliser parts 132. This aligns the needle with the axis of the tip electrode 64. Once in this position, the adjustable grip parts 114 can be driven 142 towards the syringe 14 such that they engage with the sides of the main body 16 of the syringe 14.

It will be appreciated that the right-hand grip part 114 (as shown in the drawing) will contact the main body 16 and the syringe 14 first (in this example), at which point the plate 122 will slide 150 right, as indicated by the arrow 150 in FIG. 12. Eventually, the second grip part 114 will engage the opposite side of the syringe 14 main body 16 and the syringe 14 will be clamped between the grip parts 114. At this point, the locking ring 128 can be tightened to lock plate 122 relative to its mount 126 and now the plate 122 can be moved 152 axially with the needle 24 centralised on the axis 140 of the device 100.

This configuration is shown in FIG. 13, whereby the grip parts 114 have engaged the main body 16 of the syringe 14 and the plate 122 has moved 150 so that the needle 24 is aligned with the axis 140.

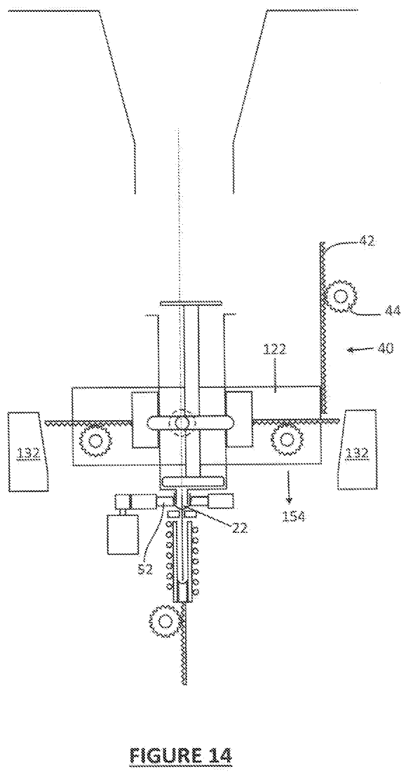

Referring now to FIG. 14 of the drawings, the centraliser parts 132 have been moved apart and this enables the plate 122 to be driven 154 downwards (in the drawings), for example using a rack 42 and pinion 44 actuator arrangement 40. The plate 122 can be driven down 154 until the hub 22 engages the annular hub grip 52 in the manner previously described, and the needle destruction procedure and needle/syringe separation procedure can proceed in the manner previously described with reference to FIGS. 1 to 10 above.

It will be appreciated that the invention is not restricted to the details of the foregoing embodiments, which are merely exemplary of the invention. For example, the linear actuators have been described as being rack and pinion devices, they could equally be pulley belt type arrangements, linear actuators or worm screws.

1. A syringe processing apparatus for syringe disposal, the syringe comprising a main body and a hypodermic needle affixed thereto, the main body comprising a spigot to which a hub that supports a needle part of the hypodermic needle is affixed, the syringe processing apparatus comprising:
a needle destruction assembly for destroying the needle part of the hypodermic needle, the needle destruction assembly comprising a conductive clamping electrode adapted to clamp the needle part of the hypodermic needle at a point between the hub and its tip, a conductive tip electrode for contacting the tip of the needle part of the hypodermic needle, the tip electrode being driven to move coaxially with an axis of the needle part of the hypodermic needle, means for applying a voltage between the clamping electrode and the tip electrode as the tip electrode is advanced towards the hub, such that resistive heating of the needle occurs in conjunction with axial compression exerted by the tip electrode on the needle, which softens/melts the tip of the needle, thereby blunting and sterilising the needle tip,
the apparatus further comprising:
a cradle for supporting and holding the main body and being driven to move the main body along a locus that is parallel with the said axis; and
a hub grip assembly for gripping the hub of the hypodermic needle, which comprises an annular grip part adapted to engage the hub of the hypodermic needle when the cradle is driven along the said axis, wherein
the cradle and hub grip assembly are relatively moveable so as to separate the hub of the hypodermic needle from the spigot of the main body.

2. The syringe processing apparatus of claim 1, wherein the needle destruction assembly further comprises a containment cylinder, which surrounds the needle part of the hypodermic needle, and within which the tip electrode is arranged to move.

3. The syringe processing apparatus of claim 2, wherein the dimensions of the containment cylinder prevent or inhibit the needle part of the hypodermic needle from bowing or buckling as the axial compressive force is applied thereto.

4. The syringe processing apparatus of claim 2, wherein the containment cylinder comprises a heater coil.

5. The syringe processing apparatus of claim 2, further comprising an induction coil surrounding the needle, which carries an electric current that induces, in use, a current in the needle, whereby resultant eddy current heating of the needle can be used to heat and/or melt the needle part of the hypodermic needle.

6. The syringe processing apparatus of claim 1, wherein the voltage applied between the tip electrode and the clamping electrode comprises any one or more of the group comprising: a DC voltage; an AC voltage; and an RF voltage.

7. The syringe processing apparatus of claim 1, further comprising a control/feedback system for coordinating the application of the voltage and the axial compression force.

8. The syringe processing apparatus of claim 7, wherein the control/feedback system comprises a high-speed feedback circuit adapted, in use, to control the applied voltage and displacement of the tip electrode so as to maintain the tip electrode in contact with the tip, whilst maintaining the current within the needle, between the clamping and tip electrodes, within specified parameters.

9. The syringe processing apparatus of claim 1, wherein the tip electrode comprises a formation for centralising the tip of the needle part of the hypodermic needle with the centre of the tip electrode.

10. The syringe processing apparatus of claim 1, wherein either or both of the tip electrode and cradle is/are driven to move coaxially with an axis of the needle part of the hypodermic needle by way of any one or more of the group comprising: a pulley belt arrangement; a rack and pinion; a worm screw; and a linear actuator.

11. The syringe processing apparatus of claim 1, wherein the cradle comprises a main body, which is shaped to receive the syringe main body, the shape of the main body comprising end abutments, which engage the distal and proximal ends of the syringe main body, thereby preventing or inhibiting axial displacement of the syringe relative to the cradle.

12. The syringe processing apparatus of claim 1, wherein the cradle comprises means for adjusting the cradle configuration such that the hypodermic needle automatically aligns with a datum line, which datum line is, or can be be brought into alignment with, the said axis.

13. The syringe processing apparatus of claim 12, comprising a centraliser, which comprises a tapered aperture that guides the hypodermic needle as a syringe is placed into the syringe processing apparatus, the taper being configured to cause the needle part of the hypodermic needle to align with the said axis.

14. The syringe processing apparatus of claim 12, wherein the cradle comprises adjustable grip means, which grips the syringe main body, the adjustable grip means comprising two or more grip parts that can be moved into engagement with they syringe main body, the grip parts being mounted on a support, which can slide to accommodate any offset between an axis of the syringe main body and the axis of the needle, and means for locking the support in position once the axis of the needle part of the hypodermic needle with the axis of the syringe processing apparatus.

15. The syringe processing apparatus of claim 1, wherein the hub grip assembly grips the hub of the hypodermic needle by way of an annular grip part adapted to engage the hub of the hypodermic needle when the cradle is driven dong the said axis, the annular grip part comprises a resiliently deformable annulus, which frictionally engages the hub as it is axially inserted into it.

16. The syringe processing apparatus of claim 1, wherein the cradle and hub grip assembly are relatively moveable by way of axial or reciprocating displacement of the cradle relative to the hub grip.

17. The syringe processing apparatus of claim 1, wherein the cradle and hub grip assembly are relatively moveable by way of rotational or oscillatory displacement about the said axis of the cradle relative to the hub grip.

18. The syringe processing apparatus of claim 17, when dependent on claim 16, wherein rotation/oscillation of the hub relative to the spigot is coordinated with axial/reciprocating displacement of the hub relative to the spigot.

19. The syringe processing apparatus of claim 18, further comprising a controller for coordinating the rotation/oscillation of the hub relative to the spigot with the axial/reciprocating displacement of the hub relative to the spigot, the controller being configured to measure and/or control the relative forces and/or relative displacements.

20. The syringe processing apparatus of claim 19, further comprising a logic circuit, which attempts to disconnect the hub from the spigot via various methods, including: axial displacement only; rotational displacement only; axial displacement+rotational oscillation; axial displacement+rotation; and axial rotation+linear displacement.
